(19) United States
(12) Patent Application Publication
(10) Pub. No.: US 2016/0361245 A1
(43) Pub. Date: Dec. 15, 2016

(54) COSMETIC COMPOSITIONS OF MITOCHONDRIALLY TARGETED ANTIOXIDANTS

(71) Applicant: MITOTECH, S.A., Luxembourg (LU)

(72) Inventors: VALERIYA VLADIMIROVNA PELEKH, Moscow (RU); Valeriy Vasilieveech Lyandin, Moscow (RU); Anna Vladimirovna Shibaeva, Puskino (RU); Maxim Vladimirovich Skulachev, Moscow (RU); Lawrence T Friedhoff, River Vale, NJ (US)

(73) Assignee: MITOTECH SA, Luxembourg (LU)

(21) Appl. No.: 15/121,600

(22) PCT Filed: Feb. 25, 2015
(86) PCT No.: PCT/IB2015/001030

§ 371 Date: Aug. 25, 2016

Related U.S. Application Data
(60) Provisional application No. 61/944,205, filed on Feb. 25, 2014.

(51) Int. Cl. A61K 8/55; A61K 8/92; A61K 8/97; A61K 8/81; A61K 8/891; A61Q 19/08; A61K 8/73

(52) U.S. Cl. CPC A61K 8/55; A61Q 19/08; A61K 8/922; A61K 8/73; A61K 8/733; A61K 8/8111; A61K 8/891; A61K 8/97; A61K 2800/594; A61K 2800/54

(57) ABSTRACT

Dislosed are compositions comprising SkQ1 for protection of the skin from damage caused by free radicals and/or re active oxygen species.

### SPHERE OF APPLICATION

This invention relates to biology and medicine and in particular to cosmetology, and may be used for manufacturing a composition for protecting the skin from damage caused by free radicals and/or active forms of oxygen.

### TECHNICAL FIELD OF INVENTION

Mitochondrially targeted antioxidants (MTA) are one of the most promising classes of anti-age products for use in various products including cosmetic products. However, various MTAs, including those in the SkQ class, in multi-component liquid mixtures are not greatly stable over long periods of storage (over 30 days). The prior art refers to a number of versions of stable compositions of MTA (see for example application WO2012167236), but the number of compositions discovered in technical terms is extremely limited for application as a finished cosmetic product. Therefore, the creation of a finished cosmetic product containing a MTA, and particularly SkQ-class compounds, with sufficient storage stability is an unresolved technical task.

Definitions

1. Mitochondrial antioxidant—compound with general formula 1:

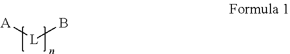

Where A is an antioxidant with general formula 2:

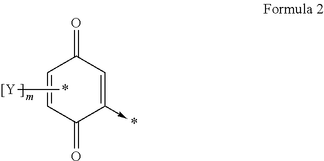

Where Y where m is an integer from 1 to 3; Y refers to single or various substitutes that constitute a lower alkyl or lower alkoxy, including the methyl or methoxy groups;

and/or its restored form

L is a linker group, that is:

a) a straight or branched hydrocarbon chain, not necessarily replaced with one or more substitutes, and where necessary containing one or more double or triple bonds; or

b) a natural isoprenoid chain;

n is an integer from 1 to 20;

B is a Skulachev ion Sk:

Sk⁺Z⁻

where Sk is a lipophilic cation; Z is a pharmacologically acceptable anion

2. Stable composition for cosmetic use is a composition within which the formula 1 compound retains sufficient stability over at least 30 days when stored at a temperature of +5° C.

3. Emollients are substances from various chemical groups, hydrophilic and lipophilic, capable of softening the skin.

4. Multifunctional components are components of a cosmetic product that fulfil more than one function.

Names and Description of Some Components of Finished Cosmetic Products and Their Manufacturers:

(manufacturers shown in square brackets)

Application of the invention is not limited to use of the specific components shown (trade names) or specifically these manufacturers. The composition of components is significant, and the manufacturer and trade product may be different. Manufacturers and trade names are shown as proof of possibility of applying the invention and as an indication for qualified specialists of what ingredients (regardless of their trade names and manufacturers) were used in the present invention.

Biocol LG (Ceratonia Siliqua Gum, Pectin, Algin) [Manufacturer: Biogenico Worldwide].

Biocol AX (Pectin, Xanthan Gum) [Biogenico Worldwide].

Makimousse12 (Sodium Polyacrylate Starch) [Daito Kasei].

Methocel 40-202 (Hydroxypropyl Methylcellulose) [DOW].

Nexbase 2006 FG (Hydrogenated Polydecene) [Neste Oil Oyj].

Silsoft 034 (Caprylyl Methicone) [Momentive Performance Materials].

Sensolene (Ethyl hexyl Olivate) [B@TCompany].

Lexfeel 7 (Neopentyl Glycol Diheptanoate) [Inolex].

Biolin/P (Inulin, Alpha-glucan Oligosaccharide) [Goya Ingredients].

Drieline1s (Sorbitol, Yeast Extract) [LucasMeyer cosmetics].

Peptiskin (Arginine/lysine Polypeptide) [Solabia Group].

Gatuline In-Tense (Caprylic/Capric Triglyceride, Spilanthes Acmella Flower Extract) [Gattefosse].

Vegetensor (Pisum Sativum Extract, Sclerotium Gum) [Alban Muller International].

Argireline (Aqua, Acetyl Hexapeptide-8) [Lipotec Group].

Ecoffea (Aqua, Glycerine, Coffea Arabica (Coffee) Seedcake Extract) [Chemyunion Quimica LTDA].

Dermocea (Aqua, Sucrose, Meristotheca Dakarensis Extract, Jania Rubens Extract) [Gelyma].

RonaCare Cyclopeptide-5 (Aqua, Alcohol, Lecithin, Ectoin, Cyclotetrapeptide-24 Aminocyclohexane Carboxylate) [Merck KGaA].

RonaCare Luremin (Dihydroxymethylchromone, Sorbitol) [Merck KGaA].

RonaFlair MTU (Bismuth Oxychloride) [Merck KGaA].

Colorona Oriental Beige (Mica, C177891 (TiO2), C177491 (FeO)) [Merck KGaA].

Colorona Red Gold (Mica, Titanium Dioxide, Iron Oxide) [Merck KGaA].

Microcare IT (Methylchloroisothiazolinone, Methylisothiazolinone) [Thor Sp].

Dipotassium Glycyrrhizate [Selco].

Ethyl Ascorbic Acid [Selco].

Allantoin [DSM]

D-Panthenol [DSM]

Sodium Ascorbyl Phosphate. [BASF]

Propylene Glycol. [INEOS].

Hydrolite-5 (Pentylene Glycol) [Symrise].

Sensiva SC 50 (Ethyl hexyl glycerine) [Schulke@Mayr].

### DESCRIPTION OF INVENTION

The present invention provides a composition and manufacturing process for finished multi-component cosmetic products containing MTAs. Overall, the key aspect of the invention is stable cosmetic composition containing mitochondrial antioxidant, and at least one additional component from the following list: gel-forming component, emollient, multi-functional component with antimicrobial activity, additional component—active cosmetic ingredient.

Examples (not limited to scope of claims under the present invention) are the following compositions:

| | |
|---|---|
| Component | Concentration |

Composition 1

| | |
|---|---|
| SkQ1 | 1 nM-1 mM |
| Gel-forming agent Biocol LG (carob tree gum, | 0.2-5% |
| pectin, sodium alginate) | |
| Hydrogenated polydecene | 0.01-40% |
| Caprylyl methicone | 0.01-40% |
| Ethyl hexyl glycerine | 0.1-2% |
| Pentylene glycol | 0.5-10% |
| Propylene glycol | 1-50% |
| Gatuline In-Tense | 0.1-5% |

Composition 2

| | |
|---|---|
| SkQ1 | 10 mcM |
| Gel-forming agent Biocol LG (carob tree gum, | 2.9% |
| pectin, sodium alginate) | |
| Hydrogenated polydecene | 0.5% |
| Caprylyl methicone | 0.5% |
| Ethyl hexyl glycerine | 0.3% |
| Pentylene glycol | 5% |
| Propylene glycol | 3% |
| Gatuline In-Tense | 2% |

Composition 3

| | |
|---|---|
| SkQ1 | 10 mcM |
| Gel-forming agent Biocol LG (carob tree gum, | 2.9% |
| pectin, sodium alginate) | |
| Hydrogenated polydecene | 0.5% |
| Caprylyl methicone | 0.5% |
| Ethyl hexyl glycerine | 0.3% |
| Pentylene glycol | 5% |
| Propylene glycol | 3% |
| Gatuline In-Tense | 2% |
| Microcare IT (methylchloroisothiazolinone | 0.1% |
| (and) methylisothiazolinone) | |

Composition 4 (on basis of exp. of sample 1)

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Benzalkonium chloride | 0.01% |

Composition 5

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Hydrogenated polydecene (Nexbase) | 1% |

Composition 6

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 1% |

Composition 7

| | |
|---|---|
| SkQ1 | 0.1-1,000 mcM |
| Biocol MG | 2.9% |
| Propylene glycol | 3% |
| Pentylene glycol | 5% |
| Ethyl hexyl glycerine | 0.3% |
| D-panthenol | 0.75% |

Composition 8

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Neopentyl glycol diheptanoate (Lexfeel 7) | 1% |

Composition 9

| | |
|---|---|
| SkQ1 | 0.1-1,000 mcM |
| Sodium Polyacrylate Starch (Makimousse 12) | 1% |
| Propylene glycol | 3% |
| Pentylene glycol | 5% |
| Ethyl hexyl glycerine | 0.3% |

Composition 10 (on base of exp. of example 2)

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Caprylic/Capric triglyceride (and) Spilanthes | 2% |
| Acmella Flower Extract (Gatuline In-Tense) | |

Composition 11

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Aqua (and) Acetyl Hexapeptide-8 (Argireline) | 5% |

Composition 12

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Aqua (and) Glycerine (and) Coffea Arabica | 4% |
| (Coffee) Seedcake Extract (Ecoffea) | |

Composition 13

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Sorbitol (and) Methylchromone | 3% |
| (RonaCareLuremin) | |

Composition 14

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Bismuth Oxychloride (RonaFlair MTU) | 2% |

Composition 15

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Mica (and) Titanium Dioxide (and) Iron Oxide | 0.6% |
| (Colorona Red Gold) | |

Composition 16

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Aqua (and) Acetyl Hexapeptide-8 (Argireline) | 5% |
| Microcare IT (methylchloroisothiazolinone | 0.1% |
| (and) methylisothiazolinone) | |

Composition 17 (on basis of exp. of example 3)

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |
| Gatuline In-Tense | 1.8% |
| RonaCare Luremin | 3% |

Composition 18

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |
| Gatuline In-Tense | 1.8% |
| Ecoffea | 4% |

Composition 19

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |
| Argireline | 5% |
| RonaCare Luremin | 3% |

Composition 20

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |
| Argireline | 5% |
| Ecoffea | 4% |

Composition 21

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |
| Ecoffea | 4% |
| RonaCare Luremin | 3% |

Composition 22

| | |
|---|---|
| SkQ1 | 0.1-1,000 mcM |
| Methocel 40-202 (hydroxypropyl methyl | 2% |
| cellulose) | |
| Caprylyl methicone (Silsoft 034) | 0.5% |
| Hydrogenated polydecene (Nexbase) | 0.5% |
| Propylene glycol | 3% |
| Pentylene glycol | 5% |
| Ethyl hexyl glycerine | 0.3% |
| Gatuline In-Tense | 1.8% |
| RonaCare Luremin | 3% |

Composition 23

| | |
|---|---|
| SkQ1 | 0.1-1,000 mcM |
| Methocel 40-202 (hydroxypropyl methyl | 2% |
| cellulose) | |
| Caprylyl methicone (Silsoft 034) | 0.5% |
| Hydrogenated polydecene (Nexbase) | 0.5% |
| Propylene glycol | 3% |
| Pentylene glycol | 5% |
| Ethyl hexyl glycerine | 0.3% |
| Gatuline In-Tense | 1.8% |
| Ecoffea | 4% |

Composition 24

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Gatuline In-Tense | 1.8% |
| RonaCare Luremin | 3% |

Composition 25

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Gatuline In-Tense | 1.8% |
| Ecoffea | 4% |

Composition 26

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Argireline | 5% |
| RonaCare Luremin | 3% |

Composition 27

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Argireline | 5% |
| Ecoffea | 4% |

Composition 28

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Ecoffea | 4% |
| RonaCare Luremin | 3% |

Composition 29

| | |
|---|---|
| SkQ1 | 0.1-1,000 mcM |
| Caprylyl methicone (Silsoft 034) | 0.5% |
| Hydrogenated polydecene (Nexbase) | 0.5% |
| Biocol LG | 2.9% |
| Propylene glycol | 3% |
| Pentylene glycol | 5% |
| Ethyl hexyl glycerine | 0.3% |
| Gatuline In-Tense | 1.8% |
| RonaCare Luremin | 3% |

Composition 30

| | |
|---|---|
| SkQ1 | 0.1-1,000 mcM |
| Caprylyl methicone (Silsoft 034) | 0.5% |
| Hydrogenated polydecene (Nexbase) | 0.5% |
| Biocol LG | 2.9% |
| Propylene glycol | 3% |
| Pentylene glycol | 5% |
| Ethyl hexyl glycerine | 0.3% |
| Gatuline In-Tense | 1.8% |
| Ecoffea | 4% |

Composition 31

| | |
|---|---|
| SkQ1 | 0.1-1,000 mcM |
| Caprylyl methicone (Silsoft 034) | 0.5% |
| Hydrogenated polydecene (Nexbase) | 0.5% |
| Biocol LG | 2.9% |
| Propylene glycol | 3% |
| Pentylene glycol | 5% |
| Ethyl hexyl glycerine | 0.3% |
| Argireline | 5% |
| RonaCare Luremin | 3% |

Composition 32

| | |
|---|---|
| SkQ1 | 0.1-1,000 mcM |
| Caprylyl methicone (Silsoft 034) | 0.5% |
| Hydrogenated polydecene (Nexbase) | 0.5% |
| Biocol LG | 2.9% |
| Propylene glycol | 3% |
| Pentylene glycol | 5% |
| Ethyl hexyl glycerine | 0.3% |
| Argireline | 5% |
| Ecoffea | 4% |

Composition 33

| | |
|---|---|
| SkQ1 | 0.1-1,000 mcM |
| Caprylyl methicone (Silsoft 034) | 0.5% |
| Hydrogenated polydecene (Nexbase) | 0.5% |
| Biocol LG | 2.9% |
| Propylene glycol | 3% |
| Pentylene glycol | 5% |
| Ethyl hexyl glycerine | 0.3% |
| RonaCare Luremin | 3% |
| Ecoffea | 4% |

Composition 34

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |
| Argireline | 5% |
| RonaCare Luremin | 3% |
| Ecoffea | 4% |

Composition 35

| | |
|---|---|
| SkQ1 | 0.1-1,000 mcM |
| Methocel 40-202 (hydroxypropyl methyl | 0.2% |
| cellulose) | |
| Propylene glycol | 3% |
| Pentylene glycol | 5% |
| Ethyl hexyl glycerine | 0.3% |

Composition 36 (on basis of exp. of example 4)

| | |
|---|---|
| SkQ1 | 0.1-1,000 mcM |
| Methocel 40-202 (hydroxypropyl methyl | 2% |
| cellulose) | |
| Propylene glycol | 3% |
| Pentylene glycol | 5% |
| Ethyl hexyl glycerine | 0.3% |

Composition 37

| | |
|---|---|
| SkQ1 | 0.1-1,000 mcM |
| Biocol LG | 2.5% |
| Propylene glycol | 3% |
| Pentylene glycol | 5% |
| Ethyl hexyl glycerine | 0.3% |

Composition 38

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Caprylyl methicone (Silsoft 034) | 0.5% |
| Hydrogenated polydecene (Nexbase) | 0.5% |
| Additional compositions on basis of exp. of | |
| example 1 | |

Composition 39

| | |
|---|---|
| SkQ1 | 0.1-1,000 mcM |
| Biocol LG | 2.9% |
| Propylene glycol | 3% |
| Pentylene glycol | 5% |
| Ethyl hexyl glycerine | 0.3% |
| Dipotassium glycyrrhizate | 1% |

Composition 40

| | |
|---|---|
| SkQ1 | 0.1-1,000 mcM |
| Sodium Polyacrylate Starch (Makimousse 12) | 1% |
| Propylene glycol | 3% |
| Pentylene glycol | 5% |
| Ethyl hexyl glycerine | 0.3% |

Other examples of compositions are compositions according to the table shown above, in which Biocol LG is replaced with Biocol AX. The experiment conducted showed that SkQ retrains its stability with such replacement.

Other examples of compositions are compositions containing 0.1-1,000 mcM of SkQ1 and Biocol of any type without emollients.

Another aspect of this invention is the method of manufacturing compositions containing a mitochondrial antioxidant. The said method includes the following procedures: addition to water of gel-forming agent (at temperature suitable for the gel-forming agent specifically chosen), mixing (for certain gel-forming agents after swelling, for certain gel-forming agents after the required pH value is reached), introduction of remaining active ingredients, in which case the oil-soluble active ingredients should preferably be introduced together with the hydrophobic emollients, and the mitochondrial antioxidant is introduced last.

### EXPERIMENTAL EXAMPLES

The experimental samples shown below illustrate possible ways of applying the invention and do not in any measure limit the scope of claims for this invention.

All experiments concerning stability of the cosmetic components tested are conducted under a single plan. The composition was prepared by adding gel-forming agent to water at the corresponding temperature and pH. Depending on the type of gel-forming agent with mixing or withholding necessary to swell the component and/or achieve the specific pH values. Then, after mixing, the remaining components (oil-soluble active ingredients, together with hydrophobic emollients) and the mitochondrial antioxidant are added.

The mixture was then kept in darkness at different temperatures, and the mitochondrial antioxidant content levels were determined using the LCMS/MS method on a Waters TQD chromatographic mass spectrometer.

The quantitative analysis was conducted using the UPLC-MS/MS method with SkQ-d15 of a known concentration as the internal standard and 3-chlorperbenzoic acid as the oxidising agent. The analysis was conducted in accordance with the method shown in the PCS for Visomytin eye drops.

1 analytical sample was prepared from each sample (extraction of 1 ml of methanol 12 hours, thermoshaker (27° C. 1400 rpm), centrifuging, preparation of analytical samples with dilution 5 times).

Each standard sample was injected 6 times before every nine injections of analytical samples. S/Sstd for standard samples were averaged at 45 injections.

### Experimental Example 1

### File Cos16-Cos29

The basic composition (BC1) used in this experiment was a composition with the following ingredients:

Biocol LG—2.9%

Propylene glycol—3%

Pentylene glycol—5%

Ethyl hexyl glycerine—0.3%

SkQ1—2 mcM

The following versions of compositions were examined:

| Compo- | Compo- | |
|---|---|---|
| sition | sition | |
| number | code | Ingredient |
| 1 | Cos16 | BC1 + benzalkonium chloride 0.01% |
| 2 | Cos17 | BC1 + hydrogenated polydecene (Nexbase) 1% |
| 3 | Cos18 | BC1 + caprylyl methicone (Silsoft 034) 1% |
| 4 | Cos19 | BC1 + ethyl hexyl olivate (Sensolene) 1% |
| 5 | Cos20 | BC1 + D-Panthenol 0.75% |
| 6 | Cos21 | BC1 + neopentylglycol diheptanoate (Lexfeel 7) 1% |
| 7 | Cos22 | BC1 + inulin in mixture with α-glucane |
| | | oligosaccharide (Biolin/P) 5% |
| 8 | Cos23 | BC1 + Sorbitol in mixture with yeast extract |
| | | (Drieline) 1% |
| 9 | Cos24 | BC1 + ethyl ascorbic acid 0.1% |
| 10 | Cos25 | BC1 + dipotassium glycyrrhizate 1% |
| 11 | Cos26 | BC1 + allantoin 0.3% |
| 12 | Cos27 | BC1 + ethyl ascorbic acid 0.5% |
| 13 | Cos28 | BC1 + sodium ascorbylphosphate 0.5% |
| 14 | Cos29 | Sodium Polyacrylate Starch (Makimousse 12) 1% + |
| | | propylene glycol 3% + pentylene glycol 5% + ethyl |
| | | hexyl glycerine 0.3% + SkQ1 |

0-point: Samples of cosmetics after manufacture and weighing were stored at −78 . . . -80° C. in dark plastic bottles 2 ml in volume until the analysis was conducted.

Time points: Samples of cosmetics after manufacture and weighing were stored for a set number of months at 37° C. in dark plastic bottles 2 ml in volume then stored at −78 . . . -80° C. until the analysis was conducted.

Test preparation: Heating at room temperature (30 min), extraction with methanol (1 ml, vortex, thermoshaker 1.5 hours 1,200 rpm), centrifuging (15 min 13,400 rpm)

Numerical Material:

| | | uG/mL | | | m(cos), | time, | | |
|---|---|---|---|---|---|---|---|---|
| | Med | Ave | AveDev | AveDev % | mg | month | uG/g | AveDev |
| Cos16 | 0.0493 | 0.0499 | 0.0010 | 2.1% | 108.68 | 0.0 | 0.459 | 0.009 |
| | 0.0378 | 0.0379 | 0.0002 | 0.6% | 104.37 | 1.0 | 0.363 | 0.002 |
| | 0.0355 | 0.0358 | 0.0005 | 1.3% | 96.33 | 2.0 | 0.371 | 0.005 |
| | 0.0311 | 0.0312 | 0.0004 | 1.1% | 92.17 | 3.0 | 0.338 | 0.004 |
| | 0.0323 | 0.0326 | 0.0006 | 1.8% | 106 | 4.0 | 0.308 | 0.006 |
| | 0.0307 | 0.0310 | 0.0004 | 1.4% | 105.35 | 5.0 | 0.294 | 0.004 |
| | 0.0284 | 0.0285 | 0.0002 | 0.7% | 104.65 | 6.0 | 0.272 | 0.002 |
| | 0.0265 | 0.0265 | 0.0002 | 0.7% | 93.57 | 7.0 | 0.283 | 0.002 |
| | 0.0247 | 0.0247 | 0.0001 | 0.6% | 91.99 | 8.0 | 0.269 | 0.002 |
| Cos17 | 0.0225 | 0.0228 | 0.0004 | 1.6% | 91.58 | 0.0 | 0.249 | 0.004 |
| | 0.0214 | 0.0215 | 0.0003 | 1.5% | 105.19 | 1.0 | 0.204 | 0.003 |
| | 0.0214 | 0.0213 | 0.0002 | 0.9% | 110.66 | 2.0 | 0.193 | 0.002 |
| | 0.0154 | 0.0154 | 0.0000 | 0.0% | 110.39 | 3.0 | 0.139 | 0.000 |
| | 0.0167 | 0.0166 | 0.0003 | 1.6% | 96.15 | 4.0 | 0.172 | 0.003 |
| | 0.0143 | 0.0143 | 0.0001 | 0.7% | 91.22 | 5.0 | 0.156 | 0.001 |
| | 0.0139 | 0.0140 | 0.0001 | 0.8% | 109.17 | 6.0 | 0.128 | 0.001 |
| | 0.0138 | 0.0140 | 0.0005 | 3.3% | 111.5 | 7.0 | 0.125 | 0.004 |
| | 0.0143 | 0.0142 | 0.0002 | 1.7% | 108.9 | 8.0 | 0.130 | 0.002 |
| Cos18 | 0.0372 | 0.0374 | 0.0007 | 2.0% | 96.12 | 0.0 | 0.389 | 0.008 |
| | 0.0316 | 0.0314 | 0.0008 | 2.4% | 108.39 | 1.0 | 0.290 | 0.007 |
| | 0.0232 | 0.0232 | 0.0002 | 0.9% | 101.55 | 2.0 | 0.229 | 0.002 |
| | 0.0208 | 0.0208 | 0.0001 | 0.5% | 97.72 | 3.0 | 0.213 | 0.001 |
| | 0.0186 | 0.0186 | 0.0002 | 0.9% | 100.54 | 4.0 | 0.185 | 0.002 |
| | 0.0174 | 0.0174 | 0.0004 | 2.3% | 101.44 | 5.0 | 0.172 | 0.004 |
| | 0.0169 | 0.0168 | 0.0004 | 2.1% | 104.65 | 6.0 | 0.161 | 0.003 |
| | 0.0188 | 0.0185 | 0.0005 | 2.7% | 102.84 | 7.0 | 0.179 | 0.005 |
| | 0.0179 | 0.0182 | 0.0006 | 3.4% | 110.35 | 8.0 | 0.165 | 0.006 |
| Cos19 | 0.0381 | 0.0379 | 0.0004 | 1.0% | 106.13 | 0.0 | 0.358 | 0.003 |
| | 0.0353 | 0.0352 | 0.0005 | 1.4% | 105.31 | 1.0 | 0.335 | 0.005 |
| | 0.0223 | 0.0223 | 0.0002 | 0.8% | 94.35 | 2.0 | 0.236 | 0.002 |
| | 0.0223 | 0.0221 | 0.0004 | 1.8% | 97.05 | 3.0 | 0.227 | 0.004 |
| | 0.0041 | 0.0041 | 0.0001 | 2.0% | 110.82 | 4.0 | 0.037 | 0.001 |
| | 0.0150 | 0.0150 | 0.0000 | 0.2% | 100.39 | 5.0 | 0.149 | 0.000 |
| | 0.0045 | 0.0046 | 0.0000 | 1.1% | 99.97 | 6.0 | 0.046 | 0.000 |
| | 0.0096 | 0.0097 | 0.0002 | 1.6% | 103.46 | 7.0 | 0.093 | 0.002 |
| | 0.0126 | 0.0126 | 0.0003 | 2.1% | 99.27 | 8.0 | 0.127 | 0.003 |
| Cos20 | 0.0374 | 0.0373 | 0.0004 | 1.1% | 110.4 | 0.0 | 0.338 | 0.004 |
| | 0.0307 | 0.0308 | 0.0002 | 0.6% | 91 | 1.0 | 0.339 | 0.002 |
| | 0.0238 | 0.0237 | 0.0004 | 1.6% | 97.83 | 2.0 | 0.242 | 0.004 |
| | 0.0348 | 0.0349 | 0.0003 | 0.7% | 95.19 | 3.0 | 0.366 | 0.003 |
| | 0.0223 | 0.0223 | 0.0003 | 1.1% | 114.08 | 4.0 | 0.195 | 0.002 |
| | 0.0228 | 0.0228 | 0.0002 | 1.1% | 101.98 | 5.0 | 0.224 | 0.002 |
| | 0.0174 | 0.0173 | 0.0001 | 0.6% | 106.8 | 6.0 | 0.162 | 0.001 |
| | 0.0191 | 0.0191 | 0.0002 | 0.9% | 96.46 | 7.0 | 0.198 | 0.002 |
| | 0.0178 | 0.0179 | 0.0001 | 0.7% | 102.84 | 8.0 | 0.174 | 0.001 |
| Cos21 | 0.0430 | 0.0429 | 0.0009 | 2.1% | 103.54 | 0.0 | 0.414 | 0.009 |
| | 0.0418 | 0.0416 | 0.0005 | 1.3% | 92.57 | 1.0 | 0.450 | 0.006 |
| | 0.0354 | 0.0353 | 0.0003 | 0.8% | 101.06 | 2.0 | 0.349 | 0.003 |
| | 0.0454 | 0.0456 | 0.0008 | 1.7% | 106.21 | 3.0 | 0.430 | 0.007 |
| | 0.0362 | 0.0364 | 0.0006 | 1.7% | 97.27 | 4.0 | 0.374 | 0.006 |
| | 0.0431 | 0.0433 | 0.0004 | 1.0% | 110.68 | 5.0 | 0.391 | 0.004 |
| | 0.0322 | 0.0321 | 0.0006 | 1.9% | 108.25 | 6.0 | 0.296 | 0.006 |
| | 0.0339 | 0.0340 | 0.0002 | 0.7% | 90.59 | 7.0 | 0.375 | 0.002 |
| | 0.0245 | 0.0243 | 0.0005 | 1.9% | 98.83 | 8.0 | 0.246 | 0.005 |
| Cos22 | 0.0372 | 0.0372 | 0.0007 | 1.8% | 112.87 | 0.0 | 0.330 | 0.006 |
| | 0.0203 | 0.0203 | 0.0000 | 0.0% | 105.39 | 1.0 | 0.193 | 0.000 |
| | 0.0147 | 0.0147 | 0.0000 | 0.0% | 94 | 2.0 | 0.157 | 0.000 |
| | 0.0139 | 0.0138 | 0.0002 | 1.6% | 111.08 | 3.0 | 0.124 | 0.002 |
| | 0.0087 | 0.0086 | 0.0001 | 1.5% | 102.66 | 4.0 | 0.084 | 0.001 |
| | 0.0083 | 0.0083 | 0.0000 | 0.1% | 97.32 | 5.0 | 0.085 | 0.000 |
| | 0.0060 | 0.0061 | 0.0001 | 1.7% | 97.72 | 6.0 | 0.062 | 0.001 |
| | 0.0059 | 0.0059 | 0.0001 | 1.4% | 93.14 | 7.0 | 0.063 | 0.001 |
| | 0.0063 | 0.0063 | 0.0000 | 0.3% | 98.38 | 8.0 | 0.064 | 0.000 |
| Cos23 | 0.0481 | 0.0480 | 0.0000 | 0.1% | 104.9 | 0.0 | 0.458 | 0.000 |
| | 0.0279 | 0.0277 | 0.0004 | 1.3% | 109.68 | 1.0 | 0.253 | 0.003 |
| | 0.0173 | 0.0174 | 0.0005 | 2.6% | 87.64 | 2.0 | 0.199 | 0.005 |
| | 0.0186 | 0.0188 | 0.0004 | 2.0% | 89.39 | 3.0 | 0.210 | 0.004 |
| | 0.0196 | 0.0198 | 0.0004 | 1.9% | 107.85 | 4.0 | 0.184 | 0.003 |
| | 0.0138 | 0.0139 | 0.0002 | 1.4% | 94.48 | 5.0 | 0.147 | 0.002 |
| | 0.0102 | 0.0102 | 0.0001 | 1.4% | 91.4 | 6.0 | 0.112 | 0.002 |
| | 0.0125 | 0.0126 | 0.0004 | 2.8% | 95.03 | 7.0 | 0.133 | 0.004 |
| | 0.0133 | 0.0134 | 0.0002 | 1.2% | 107.99 | 8.0 | 0.124 | 0.001 |
| Cos24 | 0.0393 | 0.0387 | 0.0010 | 2.7% | 97.47 | 0.0 | 0.397 | 0.011 |
| | 0.0197 | 0.0196 | 0.0003 | 1.5% | 111.7 | 1.0 | 0.175 | 0.003 |
| | 0.0085 | 0.0085 | 0.0001 | 1.5% | 103.49 | 2.0 | 0.082 | 0.001 |
| | 0.0153 | 0.0153 | 0.0003 | 2.0% | 112.53 | 3.0 | 0.136 | 0.003 |
| | 0.0116 | 0.0116 | 0.0001 | 0.5% | 93.66 | 4.0 | 0.124 | 0.001 |
| | 0.0096 | 0.0095 | 0.0001 | 1.0% | 111.19 | 5.0 | 0.086 | 0.001 |
| | 0.0050 | 0.0050 | 0.0001 | 1.2% | 99.02 | 6.0 | 0.051 | 0.001 |
| | 0.0065 | 0.0065 | 0.0001 | 1.4% | 111 | 7.0 | 0.059 | 0.001 |
| | 0.0053 | 0.0053 | 0.0001 | 2.8% | 91.19 | 8.0 | 0.058 | 0.002 |
| Cos25 | 0.0409 | 0.0405 | 0.0007 | 1.8% | 89.09 | 0.0 | 0.454 | 0.008 |
| | 0.0311 | 0.0307 | 0.0005 | 1.6% | 109.77 | 1.0 | 0.280 | 0.004 |
| | 0.0282 | 0.0279 | 0.0006 | 2.2% | 107.22 | 2.0 | 0.260 | 0.006 |
| | 0.0239 | 0.0239 | 0.0002 | 0.9% | 93.9 | 3.0 | 0.254 | 0.002 |
| | 0.0230 | 0.0231 | 0.0001 | 0.6% | 98.16 | 4.0 | 0.235 | 0.001 |
| | 0.0226 | 0.0226 | 0.0005 | 2.2% | 110.1 | 5.0 | 0.205 | 0.004 |
| | 0.0199 | 0.0198 | 0.0003 | 1.5% | 100.61 | 6.0 | 0.196 | 0.003 |
| | 0.0204 | 0.0205 | 0.0003 | 1.3% | 111.4 | 7.0 | 0.184 | 0.002 |
| | 0.0190 | 0.0191 | 0.0002 | 1.1% | 108.37 | 8.0 | 0.177 | 0.002 |
| Cos26 | 0.0446 | 0.0448 | 0.0005 | 1.0% | 104.55 | 0.0 | 0.429 | 0.004 |
| | 0.0226 | 0.0227 | 0.0002 | 0.8% | 99.17 | 1.0 | 0.229 | 0.002 |
| | 0.0188 | 0.0187 | 0.0002 | 1.2% | 103.84 | 2.0 | 0.180 | 0.002 |
| | 0.0171 | 0.0172 | 0.0002 | 0.9% | 113.75 | 3.0 | 0.151 | 0.001 |
| | 0.0110 | 0.0109 | 0.0002 | 2.2% | 89.97 | 4.0 | 0.121 | 0.003 |
| | 0.0106 | 0.0104 | 0.0002 | 2.2% | 103.9 | 5.0 | 0.100 | 0.002 |
| | 0.0078 | 0.0078 | 0.0001 | 1.6% | 100.73 | 6.0 | 0.078 | 0.001 |
| | 0.0080 | 0.0080 | 0.0000 | 0.0% | 111.11 | 7.0 | 0.072 | 0.000 |
| | 0.0059 | 0.0059 | 0.0000 | 0.0% | 105.86 | 8.0 | 0.056 | 0.000 |
| Cos27 | 0.0394 | 0.0404 | 0.0014 | 3.5% | 95.99 | 0.0 | 0.421 | 0.015 |
| | 0.0237 | 0.0235 | 0.0003 | 1.2% | 100.15 | 1.0 | 0.235 | 0.003 |
| | 0.0159 | 0.0159 | 0.0003 | 2.1% | 94.61 | 2.0 | 0.168 | 0.003 |
| | 0.0123 | 0.0121 | 0.0004 | 3.2% | 96.31 | 3.0 | 0.125 | 0.004 |
| | 0.0100 | 0.0101 | 0.0004 | 3.8% | 109.65 | 4.0 | 0.092 | 0.004 |
| | 0.0055 | 0.0055 | 0.0001 | 1.5% | 104.03 | 5.0 | 0.053 | 0.001 |
| | 0.0031 | 0.0032 | 0.0001 | 3.4% | 111.45 | 6.0 | 0.028 | 0.001 |
| | 0.0035 | 0.0034 | 0.0002 | 4.6% | 112.73 | 7.0 | 0.030 | 0.001 |
| | 0.0025 | 0.0025 | 0.0000 | 0.0% | 105.36 | 8.0 | 0.024 | 0.000 |
| Cos28 | 0.0454 | 0.0457 | 0.0005 | 1.1% | 114.27 | 0.0 | 0.400 | 0.004 |
| | 0.0091 | 0.0091 | 0.0001 | 0.7% | 95.23 | 1.0 | 0.096 | 0.001 |
| | 0.0050 | 0.0050 | 0.0000 | 0.6% | 107.99 | 2.0 | 0.047 | 0.000 |
| | 0.0026 | 0.0026 | 0.0001 | 3.6% | 95.14 | 3.0 | 0.027 | 0.001 |
| | 0.0020 | 0.0020 | 0.0000 | 0.9% | 97.41 | 4.0 | 0.021 | 0.000 |
| | 0.0017 | 0.0016 | 0.0001 | 3.7% | 95.45 | 5.0 | 0.017 | 0.001 |
| | 0.0012 | 0.0012 | 0.0001 | 5.3% | 104.59 | 6.0 | 0.011 | 0.001 |
| | 0.0010 | 0.0010 | 0.0000 | 3.1% | 92.12 | 7.0 | 0.011 | 0.000 |
| | 0.0013 | 0.0013 | 0.0001 | 5.6% | 108.81 | 8.0 | 0.012 | 0.001 |
| Cos29 | 0.0091 | 0.0091 | 0.0001 | 1.1% | 106.2 | 0.0 | 0.085 | 0.001 |
| | 0.0084 | 0.0083 | 0.0001 | 1.4% | 103.75 | 1.0 | 0.080 | 0.001 |
| | 0.0063 | 0.0065 | 0.0003 | 4.4% | 98.44 | 2.0 | 0.066 | 0.003 |
| | 0.0055 | 0.0055 | 0.0001 | 1.5% | 87.85 | 3.0 | 0.063 | 0.001 |
| | 0.0052 | 0.0051 | 0.0001 | 1.2% | 104.62 | 4.0 | 0.049 | 0.001 |
| | 0.0051 | 0.0051 | 0.0001 | 1.2% | 102.09 | 5.0 | 0.050 | 0.001 |
| | 0.0044 | 0.0044 | 0.0001 | 2.3% | 95 | 6.0 | 0.047 | 0.001 |
| | 0.0044 | 0.0044 | 0.0001 | 1.5% | 98.51 | 7.0 | 0.044 | 0.001 |
| | 0.0052 | 0.0053 | 0.0001 | 1.5% | 103.27 | 8.0 | 0.051 | 0.001 |

On the basis of the kinetic curves and initial losses, the time taken to reach 20% losses, and residual SkQ1 content after 1 year of storage, were calculated. The values obtained were extrapolated to real conditions of storage at room temperature (25 C) and in refrigeration temperature (4 C). The values calculated are shown in the table.

| | De- | | | | | | |
|---|---|---|---|---|---|---|---|
| | gradation | Time taken to | | | | | |
| | speed | reach 80% of | | | Residual SkQ1 | | |
| | constant at | SkQ1 from added | | | content after year | | |
| Sample | 37° C. k′, | total, months | | | of storage, % | | |
| number | month⁻¹ | 37° C. | 25° C. | 4° C. | 37° C. | 25° C. | 4° C. |
| 1 | 0.0607 | 3.7 | 8.4 | 36.2 | 48 | 73 | 93 |
| 2* | 0.0796 | | | | 21 | 36 | 49 |
| 3 | 0.0966 | 0.5 | 1.2 | 5.0 | 27 | 51 | 75 |
| 4* | 0.1947 | | | | 8 | 28 | 62 |
| 5* | 0.0927 | | | | 24 | 45 | 66 |
| 6 | 0.0509 | 2.1 | 4.9 | 20.8 | 49 | 69 | 85 |
| 7* | 0.2026 | | | | 6 | 25 | 56 |
| 8 | 0.1444 | 1.5 | 3.5 | 15.0 | 18 | 47 | 84 |
| 9 | 0.2061 | 0.3 | 0.7 | 3.2 | 7 | 29 | 67 |
| 10 | 0.0968 | 2.2 | 5.0 | 21.3 | 31 | 60 | 88 |
| 11 | 0.2283 | 0.6 | 1.5 | 6.2 | 6 | 28 | 71 |
| 12 | 0.3679 | 0.3 | 0.8 | 3.3 | 1 | 13 | 59 |
| 13 | 0.3982 | 0.2 | 0.4 | 1.8 | 1 | 11 | 54 |
| 14* | 0.0791 | | | | 7 | 12 | 17 |

*samples in which initial reduction of concentration of SkQ1 exceeds 20%.

Conclusion: Compositions 1 and 6 provide greater SkQ1 stability levels.

### Experimental example 2

### File Cos30-Cos33

The basic composition (BC2) used in this experiment was a composition with the following ingredients:

Biocol LG—2.9%

Propylene glycol—3%

Pentylene glycol—5%

Ethyl hexyl glycerine—0.3%

SkQ1—3 mcM

The experiment also used an additional mixture of components (DSK) with the following ingredients:

Caprylyl methicone (Silsoft 034) 0.5%

Hydrogenated polydecene (Nexbase) 0.5%

The following versions of compositions were examined:

| Number | Symbol | |
|---|---|---|
| of compo- | of | |
| sition | composition | Ingredients |
| 1 | 1 | BC2 + hydrogenated polydecene (Nexbase) |
| | | 1% |
| 2 | 2 | BC2 + caprylyl methicone (Silsoft 034) 1% |
| 3 | 3 | BC2 + DSK |
| 4 | 4.1 | BC2 + DSK + perfume composition |
| | | Pampleflor 34017 (Floressence) 0.1% |
| 5 | 4.2 | + perfume composition Olive Fresh 226864 |
| | | (Fragrance Oils (International) Limited |
| | | 0.1%) |
| 6 | 4.3 | BC2 + DSK + perfume composition |
| | | Freshness of Grass 10.01.0204 (AC Sense) |
| | | 0.1% |
| 7 | 5.1 Peptiskin | BC2 + DSK + Arginine/lysine polypeptide |
| | 2% | (Peptiskin) 2% |
| 8 | 5.2 Galutine | BC2 + DSK + Caprylic/Capric triglyceride |
| | In-Tuse | (and) Spilanthes Acmella Flower Extract |
| | | (Gatuline In-Tense) 2% |
| 9 | 5.3 | BC2 + DSK + Pisum sativum Extract (and) |
| | Vegenensor | Sclerotium Gum (Vegetensor) 5% |
| 10 | 5.4 Argireline | BC2 + DSK + Aqua (and) Acetyl |
| | | Hexapeptide-8 (Argireline) 5% |
| 11 | 5.5 Ecoffea | BC2 + DSK + Aqua (and) Glycerine (and) |
| | | Coffea Arabica (Coffee) Seedcake Extract |
| | | (Ecoffea) 4% |
| 12 | 5.6 | BC2 + DSK + Aqua (and) Alcohol (and) |
| | Cyclopeptide | Lecithin (and) Ectoin (and) |
| | | Cyclotetrapeptide-24 Aminocyclohexane |
| | | Carboxylate (RonaCare Cyclopeptide-5) 3% |
| 13 | 5.7 Moremine | BC2 + DSK + Sorbitol (and) |
| | | Methylchromone (RonaCare Luremin) 3%. |
| 14 | 5.8 Dermocea | BC2 + DSK + Aqua (and) Sucrose (and) |
| | | Meristotheca Dakarensis Extract (and) Jania |
| | | Rubens Extract (Dermocea) 2%. |
| 15 | 5.10 Cdermo | BC2 + DSK + Mica (and) Cl77891 (TiO2) |
| | Oriental Beige | (and) Cl77491 (FeO)(Colorona Oriental |
| | | Beige) 0.6% |
| 16 | 5.11 Ranaflair | BC2 + DSK + Bismuth Oxychloride |
| | | (RonaFlair MTU) 2% |
| 17 | 5.12 Red Gold | BC2 + DSK + Mica (and) Titanium Dioxide |
| | | (and) Iron Oxide (Colorona Red Gold) 0.6% |

Brief Description:

Experiment start date: 27.07.2012

0-point: Samples of cosmetics after manufacture and weighing were stored at −78 . . . -80° C. in dark plastic bottles 2 ml in volume until the analysis was conducted.

3.5 months, 60° C.: Samples of cosmetics after manufacture and weighing were stored for 3.5 months at 60° C. in dark plastic bottles 2 ml in volume, then stored at −78 . . . -80° C. until the analysis was conducted.

Test preparation: Heating at room temperature (30 min), extraction with methanol (1 ml, vortex, thermoshaker 1 hour 1,200 rpm), centrifuging (15 min 13,400 rpm)

Numerical Data

| | 27.07.12 | | | | | | | | | | |
|---|---|---|---|---|---|---|---|---|---|---|---|
| | 0 point | | | | | 3.5 months, 60 C. | | | | | |
| | 2 | | | | | 4 | | | | | |
| Composition | seriesID | vial | T, uG/ml | m, mg | iG/g | seriesID | vial | T, uG/ml | m, mg | uG/g | Residue, % |
| 1 | cos030 | a011 | lost | 95.80 | — | cos031 | h081 | 0.0279 | 105.17 | 0.266 | — |
| 2 | | b021 | 0.6786 | 104.32 | 6.505 | | i091 | 0.0082 | 92.61 | 0.088 | 1.2% |
| 3 | | c031 | 0.4781 | 91.30 | 5.237 | | j101 | 0.0066 | 105.9 | 0.062 | 1.4% |
| 4.1 | | d041 | 0.5498 | 99.57 | 5.521 | cos032 | a011 | 0.0076 | 95.64 | 0.079 | 1.4% |
| 4.2 | | e051 | 0.4554 | 105.03 | 4.336 | | b021 | 0.0130 | 106.3 | 0.122 | 2.8% |
| 4.3 | | f061 | 0.3441 | 111.16 | 3.095 | | c031 | 0.0077 | 89.22 | 0.086 | 2.2% |
| 5.1 Peptiskin 2% | | g071 | 0.5889 | 93.07 | 6.328 | | d041 | 0.0167 | 103.07 | 0.162 | 2.8% |
| 5.2 Galutine In-Tuse | | h081 | 0.5649 | 100.21 | 5.638 | | e051 | 0.0656 | 100.66 | 0.652 | 11.6% |
| 5.3 Vegenensor | | i091 | 0.4475 | 107.71 | 4.155 | | f061 | 0.0005 | 101.35 | 0.005 | 0.1% |
| 5.4 Agririlene | | j101 | 0.6717 | 101.38 | 6.625 | | g071 | 0.1048 | 114.17 | 0.918 | 15.6% |
| 5.5 Ecoffea | cos031 | a011 | 0.8353 | 110.28 | 7.574 | | h081 | 0.0396 | 106.27 | 0.373 | 4.7% |
| 5.6 Cyclopeptide | | b021 | 0.4856 | 105.34 | 4.610 | | i091 | 0.0064 | 99.29 | 0.064 | 1.3% |
| 5.7 Moremine | | c031 | 0.3792 | 97.06 | 3.906 | | j101 | 0.0204 | 97.45 | 0.209 | 5.4% |
| 5.8 Dermocea | | d041 | 0.3749 | 94.68 | 3.960 | cos033 | a011 | 0.0111 | 95.88 | 0.116 | 3.0% |
| 5.10 Cdermo Oriental Beige | | e051 | 0.5295 | 91.43 | 5.791 | | b021 | 0.0077 | 108.17 | 0.071 | 1.4% |
| 5.11 Ranaflair | | f061 | 0.7489 | 107.80 | 6.947 | | c031 | 0.1246 | 99.32 | 1.255 | 16.6% |
| 5.12 Red Gold | | g071 | 0.7832 | 101.68 | 7.702 | | d041 | 0.0509 | 91.41 | 0.557 | 6.5% |

Discussion of results: Accelerated storage of cosmetic compositions at 60° C. is not a sufficiently reliable way of studying the stability of mitochondrial antioxidants within a cosmetic product. However, with a specified level of reliability, the speed constant assessment values obtained can be used to extrapolate to real conditions of storage. The results of this extrapolation are shown in the table:

| | | 25 C. | | 4 C. | |
|---|---|---|---|---|---|
| | | Time taken | | Time taken | |
| | | to realise | | to realise | |
| | | 20% loss | Portion of PDTF | 20% loss | Portion of PDTF |
| | 60 C. | PDTF, | remaining after | PDTF, | remaining after |
| Composition | k′ month⁻¹ | months | year of storage, % | months | year of storage, % |
| 1 | 0.865203 | 3 | 40 | 13 | 81 |
| 2 | 1.229426 | 2 | 27 | 9 | 74 |
| 3 | 1.267534 | 2 | 26 | 9 | 73 |
| 4.1 | 1.21339 | 2 | 28 | 9 | 74 |
| 4.2 | 1.020196 | 2 | 34 | 11 | 78 |
| 4.3 | 1.02377 | 2 | 34 | 11 | 78 |
| 5.1 Peptiskin | 1.047184 | 2 | 33 | 10 | 77 |
| 2% | | | | | |
| 5.2 Galutine | 0.616354 | 4 | 52 | 18 | 86 |
| In-Tuse | | | | | |
| 5.3 | 1.920751 | 1 | 13 | 6 | 62 |
| Vegenensor | | | | | |
| 5.4 | 0.564688 | 4 | 55 | 19 | 87 |
| Argirelene | | | | | |
| 5.5. Ecoffea | 0.860257 | 3 | 40 | 13 | 81 |
| 5.6 | 1.222029 | 2 | 27 | 9 | 74 |
| Cyclopeptide | | | | | |
| 5.7 | 0.836553 | 3 | 41 | 13 | 81 |
| Moremine | | | | | |
| 5.8 | 1.008688 | 3 | 34 | 11 | 78 |
| Dermocea | | | | | |
| 5.10 Cdermo | 1.257537 | 2 | 26 | 9 | 73 |
| Oriental | | | | | |
| Beige | | | | | |
| 5.11. | 0.488907 | 5 | 60 | 22 | 89 |
| Ranaflair | | | | | |
| 5.12 Red | 0.750477 | 3 | 45 | 14 | 83 |
| Gold | | | | | |

Therefore, for samples 5.2, 5.4 and 5.11, we unexpectedly obtained results indicating that these compositions possibly retain more than 50% of the active substance over a year of storage at room temperature.

### Experimental Example 3

### File Cos34-Cos36

### Study of Degradation of PDTF in Ingredients of Cosmetic Compositions at 42° C. and 60° C., Time Range 10 Days

### Start of Experiment 01/03/2013

The basic composition (BC3) used in this experiment was a composition with the following ingredients:

Methocel 40-202 (hydroxypropyl methyl cellulose) 2%

Propylene glycol 3%

Pentylene glycol 5%

Ethyl hexyl glycerine 0.3%

SkQ1 1.5 mcM

The basic composition (BC4) used in this experiment was a composition with the following ingredients:

Methocel 2%

Caprylyl methicone (Silsoft 034) 0.5%

Hydrogenated polydecene (Nexbase) 0.5%

Propylene glycol 3%

Pentylene glycol 5%

Ethyl hexyl glycerine 0.3%

SkQ1 1.5 mcM

Basic composition BC5, with the following ingredients, was also used:

Biocol LG—2.9%

Propylene glycol 3%

Pentylene glycol 5%

Ethyl hexyl glycerine 0.3%

SkQ1 1.5 mcM

Basic composition BC6, with the following ingredients, was also used:

Biocol LG—2.9%

Propylene glycol 3%

Pentylene glycol 5%

Ethyl hexyl glycerine 0.3%

SkQ1 1.5 mcM

Caprylyl methicone (Silsoft 034) 0.5%

Hydrogenated polydecene (Nexbase) 0.5%

The following versions of compositions were examined:

| Number | | |
|---|---|---|
| of | Compo- | |
| compo- | sition | |
| sition | code | Ingredients |
| 1 | 1.1 | BC3 + Gatuline In-Tense 1.8% + RonaCare Luremin |
| | | 3% |
| 2 | 1.2 | BC3 + Gatuline In-Tense 1.8% + Ecoffea 4% |
| 3 | 1.3 | BC3 + Argireline 5% + RonaCare Luremin 3%. |
| 4 | 1.4 | BC3 + Argireline 5% + Ecoffea 4%. |
| 5 | 1.5 | BC3 + Ecoffea 4% + RonaCare Luremin 3%. |
| 6 | 2.1 | BC4 + Gatuline In-Tense 1.8% + RonaCare Luremin |
| | | 3%. |
| 7 | 2.2 | BC4 + Gatuline In-Tense 1.8% + Ecoffea 4%. |
| 8 | 2.3 | BC4 + Argireline 5% + RonaCare Luremin 3%. |
| 9 | 2.4 | BC4 + Argireline 5% + Ecoffea 4%. |
| 10 | 2.5 | BC4 + Ecoffea 4% + RonaCare Luremin 3%. |
| 11 | 3.1 | BC5 + Gatuline In-Tense 1.8% + RonaCare Luremin |
| | | 3% |
| 12 | 3.2 | BC5 + Gatuline In-Tense 1.8% + Ecoffea 4%. |
| 13 | 3.3 | BC5 + Argireline 5% + RonaCare Luremin 3%. |
| 14 | 3.4 | BC5 + Argireline 5% + Ecoffea 4%. |
| 15 | 3.5 | BC5 + Ecoffea 4% + RonaCare Luremin |
| | | 3%. |
| 16 | 4.1 | BC6 + Gatuline In-Tense 1.8% + RonaCareLuremin |
| | | 3%. |
| 17 | 4.2 | BC6 + Gatuline In-Tense 1.8% + Ecoffea 4%. |
| 18 | 4.3 | BC6 + Argireline 5% + RonaCare Luremin 3%. |
| 19 | 4.4 | BC6 + Argireline 5% + Ecoffea 4%. |
| 20 | 4.5 | BC6 + Ecoffea 4% + RonaCare Luremin 3%. |
| 21 | 5.1 | BC3 + Argireline 5% + RonaCare Luremin 3% + |
| | | Ecoffea 4%. |
| 22 | 5.2 | Methocel 0.2% + propylene glycol 3% + pentylene |
| | | glycol 5% + ethyl hexyl glycerine 0.3% + SkQ |
| | | (15 mcM). |

Brief Description:

Experiment Start Date: 01.03.2013

0-points: Samples of cosmetics after manufacture and weighing were stored at −78 . . . -80° C. in dark plastic bottles 2 ml in volume until the analysis was conducted.

Time points: Samples of cosmetics after manufacture and weighing were stored for 3.5 months at 42° C. or 60° C. in dark plastic bottles 2 ml in volume, then stored at −78 . . . -80° C. until the analysis was conducted.

Test preparation: Heating at room temperature (30 min), extraction with methanol (1 ml, vortex, thermoshaker), centrifuging (15 min 13,400 rpm)

Conclusion: At 60° C. more than 35% SkQ1 remained in compositions 2, 4, 5, 7, 11, 16, 17 and 21; for storage without heating (over 70% retained)—compositions 3, 11, 14, 15, 17 and 21, but in this case the highest substance content (over 0.5 mcg/g) at the initial point was observed in compositions 5, 9, 10, 12-19 and 22.

### Experimental Example 4

### File Cos42 . . .

The following versions of compositions were examined:

| Number | Symbol of | |
|---|---|---|
| of composition | composition | Ingredients |
| 3 | Methocel | Methocel 2% |
| | | Propylene glycol 3% |
| | | Pentylene glycol 5% |
| | | Ethyl hexyl glycerine 0.3% |
| | | SkQ1 15 mcM |
| 4 | Biocol 2.5% | Biocol LG 2.5%, |
| | Silsoft 034 | propylene glycol 3%, |
| | and Nexbase | pentylene glycol 5%, |
| | | ethyl hexyl glycerine 0.3%, |
| | | SkQ 15 mcM. |
| 5 | Biocol 2.9% | Biocol LG 2.9% |
| | Silsoft 034 | Propylene glycol 3% |
| | and Nexbase | Pentylene glycol 5% |
| | | Ethyl hexyl glycerine 0.3% |
| | | Ethyl hexyl glycerine 0.3%, |
| | | caprylyl methicone (Silsoft |
| | | 034) 0.5% |
| | | Hydrogenated polydecene |
| | | (Nexbase) 0.5% |
| | | SkQ1-15 mcM |

Numerical Data

In brief:

Cos042; SkQ1; Analysis Date: 27/5/2013 13:34

| | | t, | | Ave | Rel | |
|---|---|---|---|---|---|---|
| Sample | m, mg | ° C. | uG(SkQ1)/G(cosm) | Dev | Err | Symbol of composition |
| 3.1 | 103.13 | −78 | 0.982 | 0.022 | 2.3% | Methocel |
| 3.2 | 96.32 | +60 | 0.146 | 0.023 | 15.7% | |
| 3.3 | 97.25 | +42 | 0.387 | 0.040 | 10.3% | |
| 4.1 | 103.62 | −78 | 3.879 | 0.057 | 1.5% | Biocol 2.5% Silsoft 034 and Nexbase |
| 4.2 | 103.02 | +60 | 1.283 | 0.040 | 3.1% | |
| 4.3 | 103.07 | +42 | 3.356 | 0.056 | 1.7% | |
| 5.1 | 103.60 | −78 | 4.044 | 0.102 | 2.5% | Biocol Gel Biocol 2.9% Silsoft 034 Nexbase |
| 5.2 | 96.12 | +60 | 2.006 | 0.041 | 2.0% | |
| 5.3 | 95.69 | +42 | 1.839 | 0.032 | 1.7% | |

Notes
1. “T, uG/mL”—concentration determined according to result of single injection; “T(ave), uG/mL”—average concentration of PDTF; “Ave Dev., uG/mL”—average deviation in concentration of PDTF; “Rel. Err”—relative average deviation expressed as a percentage

Brief Description:

The quantitative analysis was conducted using the UPLC-MS/MS method with PDTF-d15 of a known concentration as the internal standard and 3-chlorperbenzoic acid as the oxidising agent. The analysis was conducted in accordance with the method shown in the PCS for Visomytin eye drops.

1 analytical sample was prepared from each sample (extraction of 1 ml of methanol 12 hours, thermoshaker (27° C. 1400 rpm), centrifuging, preparation of analytical samples with dilution 5 times)

### Experimental Example 4

### File Cos44 . . .

In this experiment, with accelerated storage (at temperature +60° C.), the following composition (symbol Cos44) was used:
Biocol AX (Pectin, Xanthan gum) 2.9%
Propylene glycol 3%
Caprylyl methicone (Silsoft 034) 0.5%
Ethyl hexyl glycerine (Nexbase) 0.5%
Microcare IT (methylchloroisothiazolinone (and) methylisothiazolinone)
SkQ1 10 mcM

Here follow the introductory data for this experiment.

| | |
|---|---|
| Composition identifier | Cos044 |
| Specified substances, method | SkQ1, UPLC-MS/MS |
| Matrix | Gel extract 96% EtOH |
| Method of analysis (file name) | ND 0001355-131211 dated 13.12.11 |

Results of Analysis

Composition Cos44; experiment 60° C.;
Cos044; SkQ1

| | | | T, | T(ave), | Ave Dev, | Rel |
|---|---|---|---|---|---|---|
| Sample | Date | Vial | uG/mL | uG/mL | uG/mL | Err |
| 0 point | Oct. 28, 2013 | 11 | 0.4783 | 0.4927 | 0.0185 | 3.8% |
| | | | 0.5208 | | | |
| | | | 0.4797 | | | |
| | | | 0.4863 | | | |
| | | | 0.4944 | | | |
| | | | 0.5137 | | | |
| | | | 0.4805 | | | |
| | | | 0.5250 | | | |
| | | | 0.4554 | | | |
| 1 point | Oct. 31, 2013 | 21 | 0.3261 | 0.3194 | 0.0092 | 2.9% |
| | | | 0.3139 | | | |
| | | | 0.3050 | | | |
| | | | 0.3167 | | | |
| | | | 0.3349 | | | |
| | | | 0.3284 | | | |
| | | | 0.3108 | | | |
| | | | 0.3088 | | | |
| | | | 0.3298 | | | |
| 2 point | Nov. 5, 2013 | 31 | 0.2671 | 0.2495 | 0.0087 | 3.5% |
| | | | 0.2368 | | | |
| | | | 0.2325 | | | |
| | | | 0.2661 | | | |
| | | | 0.2544 | | | |
| | | | 0.2481 | | | |
| | | | 0.2437 | | | |
| | | | 0.2487 | | | |
| | | | 0.2481 | | | |
| 3 point | Nov. 7, 2013 | 41 | 0.2477 | 0.2223 | 0.0053 | 2.4% |
| | | | 0.2236 | | | |
| | | | 0.2239 | | | |
| | | | 0.2173 | | | |
| | | | 0.2320 | | | |
| | | | 0.2254 | | | |
| | | | 0.2282 | | | |
| | | | 0.2172 | | | |
| | | | 0.2111 | | | |

In brief:

Composition Cos44; experiment 60° C.;
Cos044; SkQ1;

| | | mass, | uG(SkQ1)/ | Ave Dev, | Rel |
|---|---|---|---|---|---|
| Sample | Date | mg | G(gel) | uG/G | Err |
| 0 point | Oct. 28, 2013 | 104.64 | 4.71 | 0.18 | 3.8% |
| 1 point | Oct. 31, 2013 | 109.45 | 2.92 | 0.08 | 2.9% |
| 2 point | Nov. 5, 2013 | 109.39 | 2.28 | 0.08 | 3.5% |
| 3 point | Nov. 7, 2013 | 104.94 | 2.12 | 0.05 | 2.4% |

Notes
“T, uG/mL”—concentration determined according to result of single injection;
“T(ave), uG/mL”—average concentration of SkQ1;
“Ave Dev., uG/mL”—average deviation in concentration of SkQ1;
“Rel. Err”—relative average deviation expressed as a percentage

Conclusion: Extrapolation of the data obtained for real storage temperatures of cosmetic products indicates that 50% of substance is lost over 50-100 days of storage at 25 C or 200-400 days of storage at 4 C.

Brief Description:

The quantitative analysis was conducted using the UPLC-MS/MS method with SkQ1-d15 of a known concentration as the internal standard and 3-chlorperbenzoic acid as the oxidising agent. The analysis was conducted in accordance with the method shown in the PCS for Visomytin eye drops.

From each sample of solution, a drop was produced for 1 analytical sample.

S/Sstd for standard samples was averaged for 24 injections (no ejections). Average value of S/Sstd for standard samples, equal to 2.162+/−0.124, used for calculating SkQl concentration in solutions studied.

1. Composition for cosmetic use, comprising mitochondrial antioxidant in concentration from 1 nM to 1 mM, with condition that during storage of composition for at least 30 days at temperature in excess of 0° C., the ingredients of the composition retained not less than 70% of the initial concentration of mitochondrial antioxidant.

2. Composition according to claim 1, comprising gel-forming component

3. Composition according to claim 2, characterised in that the gel-forming components are macromolecular colloids or associated organic colloids, or aggregates formed by non-organic molecules and particles (non-organic colloids).

4. Composition according to claim 3, characterised in that the gel-forming components are synthetic polymers and co-polymers (polyethylene glycol, polyacrylic acid and polyacrylates) or cellulose and its derivatives), or natural resins (guar and xanthan gum, carrageenan etc.), or polysaccharides (starch and starch derivatives, alginates), or proteins and hydrolysed proteins, or a mixture of various gelling agents.

5. Composition according to claims 2-4, characterised in that the gel-forming component includes carob tree gum.

6. Composition according to claims 2-5, characterised in that the gel-forming component includes pectin.

7. Composition according to claims 2-6, characterised in that the gel-forming component includes sodium alginate.

8. Composition according to claims 1-7, comprising a hydrophilic emollient,

9. Composition according to claim 8, comprising a polyol-based emollient.

10. Composition according to claim 8, comprising an emollient based on water-soluble esters.

11. Composition according to claim 8, comprising an emollient based on vegetable extracts.

12. Composition according to claims 1-7, comprising a hydrophobic emollient.

13. Composition according to claim 12, characterised in that the emollient includes one or more substances from the following list: natural or synthetic fats, oils, waxes; hydrocarbons, higher carbonic acids and alcohols, silicon.

14. Composition according to claim 13, comprising hydrogenated polydecene and caprylyl methicone.

15. Composition according to claim 12, comprising caprylic/capric triglycerides.

16. Composition according to claims 1-13, comprising one or more multi-functional components with antimicrobial activity.

17. Composition according to claims 1-16, comprising an additional active substance (active ingredient).

18. Composition according to claim 17, characterised in that one of the additional active substances is acmella extract

19. Composition according to claims 1-18, characterised in that the mitochondrial antioxidant is a compound of general formula 1: 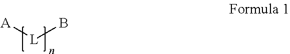
where A is an antioxidant of general formula 2: 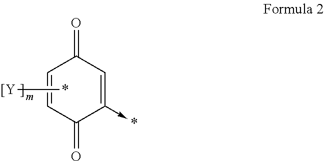
Where Y where m is an integer from 1 to 3; Y refers to single or various substitutes that constitute a lower alkyl or lower alkoxy, including the methyl or methoxy groups;
and/or its restored form
L is a linker group, that is:
a) a straight or branched hydrocarbon chain, not necessarily replaced with one or more substitutes, and where necessary containing one or more double or triple bonds; or
b) a natural isoprenoid chain;
n is an integer from 1 to 20;
B is a Skulachev ion Sk:
Sk⁺Z⁻
where Sk is a lipophilic cation; Z is a pharmacologically acceptable anion

20. Composition according to claim 22, characterised in that the mitochondrial antioxidant is a SkQ1 compound: 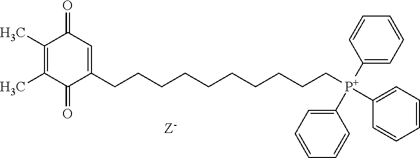 [SMILES: CC1=C(C)C(=O)C(CCCCCCCCCC[P+](c2ccccc2)(c2ccccc2)c2ccccc2)=CC1=O]
or its restored form,
where Z⁻ is a pharmacologically acceptable anion.

24. Cosmetic composition according to claim 23, characterised in that its ingredients of which include mitochondrial antioxidant SkQ1, a gelling agent (carob tree gum, pectin, sodium alginate), an emollient (hydrogenated polydecene and/or caprylyl methicone), ethyl hexyl glycerine, pentylene glycol, propylene glycol.
